(19) United States
(12) Patent Application Publication
(10) Pub. No.: US 2012/0071610 A1
(43) Pub. Date: Mar. 22, 2012

(54) REGIOFUNCTIONAL CARBON NANOTUBE BEAM AND METHOD

(76) Inventor: Nolan Walker Nicholas, South Charleston, WV (US)

(21) Appl. No.: 13/045,047

(22) Filed: Mar. 10, 2011

Related U.S. Application Data
(63) Continuation-in-part of application No. 12/854,763, filed on Aug. 11, 2010.

(51) Int. Cl. C08F 8/00; C01B 31/04; C01B 33/00; C07C 2/00; C07C 41/01; C01B 35/00; B82Y 40/00

(52) U.S. Cl. 525/331.9; 568/609; 423/448; 423/276; 423/324; 585/700; 977/848

(57) ABSTRACT

The present invention is generally directed toward a method to create an enhanced carbon nanotube spaceframe network. The spaceframe network contains an assembly of regiofunctional carbon nanotube beams by crown-to-crown connection into nodes to form a networked lattice configuration. The inventive method includes selecting crown materials and applying appropriate processing conditions which result in the production of secondary forms. The crown materials include polymers with unsaturated sites, polymeric crowns, silicon boron, poly(hydridocarbyne). The processing conditions include radical initiation, vulcanization, pyrolysis, hydroboration at unsaturation sites, using silicon bearing polymers in the Rf-CNB crowns, dissolution of silicon containing organics into the nodes and poly(hydridocarbyne). The secondary forms include cross-linked polymers, carbonized, graphitized, ceramic, diamond-like along with tailored functionalization.

### REFERENCE TO PENDING APPLICATIONS

This application claims the benefit of U.S. patent application Ser. No. 12/854,763 filed Aug. 11, 2010 entitled Regiofunctional Carbon Nanotube Beam and Method.

### REFERENCE TO MICROFICHE APPENDIX

This application is not referenced in any microfiche appendix.

### BACKGROUND OF THE INVENTION

The present invention is directed toward to carbon nanotubes, and more specifically toward regiofunctional carbon nanotube beams (Rf-CNBs) and related methods and applications.

Prior art has demonstrated the functionalization of nanotubes with more than one type of functional group, and even configurations of nanotubes with differing functionalities localized to end versus sidewall regions of the nanotubes. However, these materials have never been considered for geometrically directed self-assembly of these materials. And furthermore these materials were not designed or synthesized with or for the type of geometrical precision suitable for self-assembly applications.

Additionally, prior art has described in general terms the use of functionalized nanotubes for the self-assembly of nanotube materials. However, this disclosure in the prior art is insufficient in its teaching to lead to the present invention without the development of further inventive steps.

### SUMMARY OF THE INVENTION

The present invention satisfies the needs discussed above. The present invention is generally directed toward carbon nanotubes, and more specifically toward regiofunctional carbon nanotube beams (Rf-CNBs) and related methods and applications. More specifically, the present invention comprises the region-selective localization of the functionalizing agents to the ends versus the sidewalls to create physico-chemical differences which are advantageous for the assembly of network and membrane structures which are specifically envisioned for these materials or to specify the self-assembly of other structures assembled from RsF-CNB materials.

The following definitions will be used throughout this disclosure.

The terminal sites of the carbon nanotubes (proper) in the axial direction are referred to as the “ends” of the carbon nanotube. These ends may consist of a grapheme closure, such as a fullerene cap, or of a functional group termination consisting of non-graphenic moieties at the terminal site of the graphenic lattice and act to stabilize graphenic lattice edge. Common native terminal moieties include: hydrogen, hydroxyl, and carboxyl, ketone and other moieties.

The cylindrical surface of the nanotube is referred to as the “sidewall” of the nanotube. The term “sidewall” may be used to describe either a “pristine” sidewall which is a substantially perfect graphenic lattice without attached functional groups, or a functionalized sidewall in which functional groups have been attached to the sidewall of the nanotube.

The non-graphitic material affixed to the ends of the carbon nanotube is termed the “crown.”

“Carbon nanotubes” (“CNTs”) may refer to single-walled or multiwalled carbon nanotubes

Non-covalent functionalization may be achieved by interactions including: ionic bonding, pi-stacking, solvophobic interactions, van der Waals forces, et cetera; or combinations of these. This is in distinction to covalent functionalization wherein functional moieties are linked directly to the carbon nanotube lattice through a covalent bond.

In one aspect of the present invention, regiofunctional carbon nanotube beams are considered a novel nanomaterial structure for geometrically directed self-assembly of materials incorporating nanotube functionalities. Regiofunctional Carbon Nanotube Beams are a nanomaterial structure comprising a carbon nanotube with a mass of non-graphitic material affixed to each end of the nanotube and wherein the sidewalls of the nanotube are substantially free from this material. In this aspect, the sidewalls are functionalized (covalently or non-covalently) with a material different from the material affixed to the nanotube ends. The different regions of materials attached to the nanotube (at the ends vs. on the sidewalls) can be used to engender regiospecified physico-chemical properties which can be utilized for directed manipulation of these nanomaterials including self-assembly and other applications. These structures are useful to enable self-assembly nanotube based superstructures including: nanotube spaceframe lattices and nanotube-based fluid-permeable membranes.

In another aspect of the present invention, a process to produce regiofunctional carbon nanotube beam structures is disclosed. This process utilizes chemical moieties attached selectively to the ends and/or the sidewalls of the nanotube which differentiate the physico-chemical properties of the nanotube ends from the physico-chemical of the sidewalls to enable directed self-assembly.

Upon reading the included description, various alternative embodiments will become obvious to those skilled in the art. These embodiments are to be considered within the scope and spirit of the subject invention, which is only limited by the claims which follow and their equivalents.

### BRIEF DESCRIPTION OF THE DRAWINGS

FIG. 1 is a graphical illustration of an embodiment of a regiofunctional carbon nanotube beam of the present invention.

FIG. 2 is a schematic of the inventive process to produce regiofunctional carbon nanotube beam structures of the present invention.

FIG. 3 illustrates a two-dimensional version of a carbon nanotube spaceframe lattice of the present invention.

### DESCRIPTION OF THE PREFERRED EMBODIMENTS

The present invention is generally directed toward carbon nanotubes, and more specifically toward regiofunctional carbon nanotube beams (Rf-CNBs) and related methods and applications.

The inventive Rf-CNBs are a novel nanomaterial structure for geometrically directed self-assembly of materials incorporating nanotube functionalities. Regiofunctional Carbon Nanotube Beams are a nanomaterial structure comprising a carbon nanotube with a mass of non-graphitic material affixed to each end of the nanotube and wherein the sidewalls of the nanotube are substantially free from this material.

In an embodiment the sidewalls are functionalized (covalently or non-covalently) with a material different from the material affixed to the nanotube ends. In this embodiment, the different regions of materials attached to the nanotube (at the ends vs. on the sidewalls) can be used to engender regiospecified physico-chemical properties which can be utilized for directed manipulation of these nanomaterials including self-assembly and other applications.

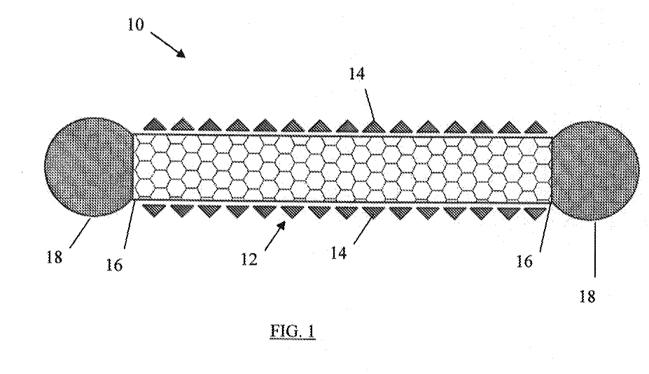

FIG. 1 illustrates an embodiment 10 of the inventive Rf-CNB of the present invention. These structures are useful to enable self-assembly nanotube based superstructures including: nanotube spaceframe lattices and nanotube-based fluid-permeable membranes. FIG. 1 illustrates a nanotube 12 having a sidewall 14, two ends 16 and one crown 18 attached to each end 16.

It should be noted that equivalent objects fabricated from nanomaterials other than carbon nanotubes which also have high aspect ratios (greater than 5, and often much greater than 5) with regiofunctionalized ends-versus-sidewalls are considered in this invention and can often be utilized for similar applications. Such materials may include regiofunctionalized carbon nanofibers, metallic nanorods and whiskers, semiconductor nanowires, etc.

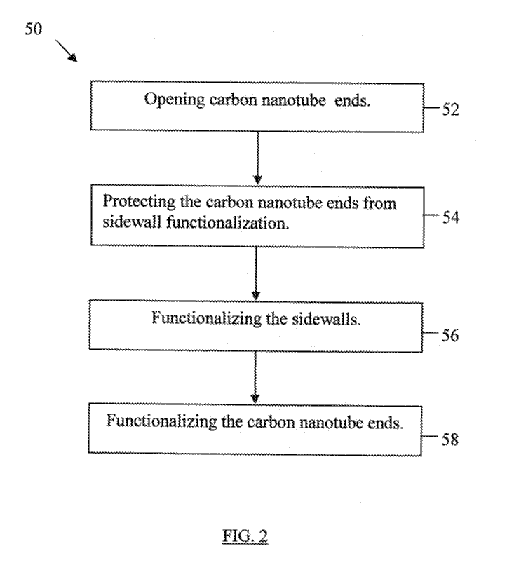

As illustrated in FIG. 2, an embodiment 50 of the inventive process to produce regiofunctional carbon nanotube beam structures is also disclosed. The process utilizes chemical moieties attached selectively to the ends and/or the sidewalls of the nanotube which differentiate the physico-chemical properties of the nanotube ends from the physico-chemical of the sidewalls to enable directed self-assembly. In generally, the inventive process includes opening carbon nanotube ends 52, protecting the ends from sidewall functionalization chemistry by chemically differentiating the open carbon nanotube ends from the nanotube sidewall 54, functionalizing the sidewalls 56, functionalizing the carbon nanotube ends which is accomplished by attaching crown to the ends 58.

It should be noted that all nanotube processing recipes will depend upon the specific quality of the nanotubes utilized. In some cases it may be advantageous to perform purification and/or annealing heat treatment steps prior to the steps outlined below to improve the quality of the product produced.

With regard to the opening of the nanotube ends, due to the harsh conditions typically required to attack pristine graphenic carbon (such as that of pristine nanotubes), this step will typically be performed first to avoid attacking other chemical moieties present (such as sidewall attached functional groups). This step “activates” the ends of the nanotube selectively by providing a high areal density of chemically distinct and reactive sites localized to the end of the nanotube. This step must be performed in such a way as to not do significant damage to the sidewalls of the nanotube (the chemical identity of the sidewalls must be maintained substantially intact).

Opening nanotube ends has been demonstrated by a variety of methods including: (1) oxidative etching, (2) mechanical milling/grinding, and (3) ultrasonication. Species demonstrated for oxidative etching include: (1) oxidizing acids, (2) hydrogen peroxide, (3) ozone, (4) potassium permanganate, (5) basic hydroxides (KOH, NaOH, NH4OH, etc.), (5) carbon dioxide, (6) singlet oxygen, etc. Often times these species are used in conjunction (e.g. acid piranha, base piranha, etc.) with each other and with specific activating conditions (reflux, ultrasonication, etc.) Base piranha (NH4OH:H2O2) and molten hydroxide treatments have been reported to be suitable for the opening of carbon nanotubes without significant damage to the sidewall structure. Base piranha is also sometimes referred to as an “RCA etch.” Increased end selectivity for end localized reaction can be obtained by applying a high frequency (1 KHz-1 GHz) high strength electric field (≧5E3 V/m [exact required value is inversely dependent upon the length of the nanotube and linearly dependent upon its dielectric environment]) during the chemical etching/opening step. For “irreversible” chemical reactions which can proceed by electron transfer (such as oxidation), the application of such a field induces a voltage differential along the axis of the applied voltage proportional to the scalar product of the length of the tube with in the direction of the applied field. A total voltage difference as small as tens of mV can be enough to have a noticeable effect on the system reactivity.

In some cases it may be advantageous to perform the opening as a two-step process wherein the nanotubes are first opened under relatively harsh conditions, then repaired by an annealing process under inert or mildly carbonizing conditions (e.g. a dilute methane atmosphere) and then re-opened under mild conditions. This provides a mechanism to open and remove the end caps, and then repair the sidewall damage often caused this process through annealing. However, though annealing may cause the ends to reconstruct into a semi-closed lattice it will not typically reform the end caps entirely so that a second, milder opening etch can fully open the CNT ends without causing significant damage to the sidewalls.

Regarding the protection of the ends from sidewall functionalization chemistry, in order to avoid functionalizing the ends of the nanotubes by the same chemistry which is used to functionalize the sidewalls of the nanotubes it is necessary to chemically differentiate ends from sidewall. This can be achieved by the introduction of chemical moieties which show distinct reactivity to that of the basal graphenic carbon lattice. The type of protective groups used will depend upon the precise chemistry chosen to functionalize the sidewalls. In some cases the chemical species introduced by opening the nanotube may provide suitable protection from the sidewall functionalization chemistry (e.g. the oxygen bearing moieties such as hydroxyls, carboxyls, quinones, and lactones introduced by oxidative etch opening will not generally react via a Diels-Alder cycloaddition route).

Alternatively it is possible to introduce other chemical groups in a second reaction which will act to protect the ends from the sidewall functionalization chemistry. The second chemical reaction is performed to link sacrificial chemical groups to the ends of the nanotube by a reversible linkage (e.g. esterification or amidation) to protect the ends of the nanotube. That is, the sidewall functionalization chemistry may also react with these sacrificial groups; however, these groups prevent the end of the nanotube itself from being functionalized by the sidewall functionalization chemistry. And, at the end of the sidewall functionalization step, these groups can be selectively detached by a chemistry which recovers the original end moiety which is then available for end-selective chemistries to introduce new chemical moieties to selectively to the ends of the nanotube (e.g. esterification/amidation linkage, graft-from polymerization, etc.) For esterification/amidation linkage reactions, the conversion of carboxyl groups to acyl chloride groups using reagents such as thionyl chloride or oxalyl chloride is a common method to increase reactivity and efficiency for this reaction.

Regarding the functionalizing of the sidewalls, such has been demonstrated by a variety of different methods including carbon radical attack. These radicals can be derived from a variety of sources including peroxide decomposition, halogen extraction, diazonium salt decomposition, etc. Additionally, a variety of chemical moieties have been attached to the sidewalls of carbon nanotubes via this method including: alkyls, aryls, phenyls, and PEG-chains.

Additional methods include Diels-Alder cycloaddition, azomethine ylide cycloaddition, reductive arylation/alkylation (Billup's Reaction), fluorination-reductive defluorination, alkoxy radical addition, nucleophilic carbene functionalization, nitrene functionalization, hydroxyl attack, esterification, amidation, Friedel-Crafts reaction and electrografting

The primary requirement of the sidewall chemistries utilized for this technology is that it result in the attachment of sidewall functional groups of the desired physico-chemical properties while not functionalizing or otherwise disrupting the physico-chemical identity of the ends of the nanotubes (which are typically protected by a suitably selected chemical moiety). Many of these reaction types are useful in conferring sites for further attachment of other functional materials (e.g. polymer chains).

Regarding the functionalizing of the ends, functionalization of the open ends can be achieved by a variety of methods including: (1) graft-to polymerization, (2) graft-from polymerization, (3) esterification linkage, (4) amidation linkage, (5) thiolation & linkage, (6) urethane linkage, et cetara; or a combination of these methods may be used. The primary criterion for end functionalization is that it create suitable chemical moieties attached selectively to the nanotube ends without detrimentally interfering with the sidewall groups.

Regarding the attaching of the crowns, attached crown can be a polymeric material (including liquid-like polymeric materials which are capable of intermixing with the crown attached to the ends of other Rf-CNBs) or non-polymeric materials such as magnetic nanoparticles. This embodiment (intermixing polymeric material) is specifically beneficial to facilitate the self-assembly process (e.g. for spaceframes).

It should be noted that despite being described as “steps” the ordering of these elements does not necessarily imply that the steps must follow this specific sequence; it is presented in this sequence as the most preferred embodiments of this technology follow this temporal sequence.

### EXAMPLES

### Example Embodiment 1

### Hydrophilic Crown

1. Base piranha etch to open ends under “mild conditions”
2. End protective sacrificial functionalization by ester linkage with polyethylene glycol (PEG)
3. Sidewall functionalization with alkyl groups (by alkyl radical attack)
4. Hydrolytic End De-Esterification—polyethylene glycol removal (protecting group removal)
5. Crown Attachment—ester linkage of methoxy polyethylene glycol (MPEG)

### Example Embodiment 2

### Hydrophobic Crown

1. Base piranha etch to open ends under “mild conditions”
2. End protective sacrificial functionalization by ester linkage with hydroxyl terminated polybutadiene (HTPB)
3. Sidewall functionalization with polyol derived hydroxyalkylalkoxy radicals or sidewall functionalization by ionic surface groups (e.g. phenyl sulfonate)
4. Hydrolytic End De-Esterification—HTPB removal (protecting group removal)
5. Crown Attachment—ester linkage of hydroxyl terminated polybutadiene (HTPB)

Regarding end linkage esterification, ester linkage to carbon nanotubes is most commonly performed utilizing carboxylic acid sites on the carbon nanotube. Often, these sites are first converted into an acyl chloride site to facilitate the ease and efficiency of the esterification reaction. Dicyclohexylcarbodiimide has also been demonstrated as an effective promoter for this reaction in nanotube systems.

Virtually any chemical with hydroxyl, carboxyl, or acyl chloride termination can be used as a linker material for this process as it is found suitable for conferring physico-chemical properties desired. The primary requirements for these species is that they be able to form ester bonds, that they not be sterically hindered from reacting with nanotube based groups, and that the chemical survive the chemical conditions used to induce esterification (typically acid or base at elevated temperature). Species of interest may include hydroxyl terminated polybutadiene, polyethylene glycol, functionalized polyethylene glycol, polypropylene glycol, poly(caprolactone), functionalized siloxanes, or any other suitable functionalized polymer including dendrimers, functionalized nanoparticles and polymerization initiators and janus nano-objects (dendrimers, nanoparticles, etc.).

Typical oxidative opening conditions create a variety of oxidized species including both carboxyl and hydroxyl moieties. Increased esterification site densities can be created by reacting the nanotubes with a di-functional ester-forming molecule such as a diol, di-acid or cyclic acid anhydride. This type of treatment will convert available hydroxyl sites into available carboxyl sites or vice versa, thereby increasing the total effective site density for further esterification reactions. Species suited to this function include ethylene glycol, oxalic acid and succinic anhydride. Multifunctional species are also useful to increase the effective linker site density at the CNT end; e.g. species such as trimesic acid, benzenetricarboxylic anhydride or glycerol can be utilized to this end.

It may be advantageous to form the crowns via a step-growth polymerization process to form polymers/oligomers of suitable size and structure. This allows the attachment of linear chains, branching chains, etc.

Regarding the sidewall functionalization by Alkyl Radicals, Lauroyl peroxide based functionalization is preferred for simplicity.

Regarding the sidewall functionalization by Alkoxy & Hydrophilic Radicals, these radicals are derived chemical species comprising polyols (e.g. ethylene glycol, glycerol, 1,3 propanediol) and hydrophilic oligomers (e.g. poly ethylene glycol) to covalently attach hydrophilic groups to the nanotube surface. This can be achieved via the suspension of CNTs into fluid compositions of these species; and then creating radical species from the hydrophilic suspending fluid (e.g. by radical abstraction of hydrogen).

This procedure represents a significant advancement in simplicity and resulting product over other procedures set out in the prior art. The use of polyols is superior to the previous literature reports of the use of alcohols since polyol based reactions guarantee at least one hydroxyl group is attached to the CNT per radical functionalization event and on average a higher percentage of hydroxyl groups attached to the CNT per functionalization event.

An example procedure is: a mixture of ethylene glycol, glycerol, and PEG (MW˜300)—in a volume ratio of ˜15:10:2, suspending a relatively high concentration of carbon nanotubes (˜1 g/L) and with a relatively large amount of benzoyl peroxide (˜6 g/L) which is allowed to decompose slowly at room temperature under inert atmosphere over the course of several days. This has been observed to generate functionalized carbon nanotubes which demonstrate improved suspension and stability into hydrophilic solvents.

In another embodiment the sidewalls are covalently functionalized by phenylsulfonate groups.

Regarding de-esterification hydrolysis, the process of de-esterification by hydrolysis is well known in the literature and in common chemical practice. Typical conditions for acid or base hydrolysis in aqueous or organic solution under elevated temperature are suited to this step.

Previous work has demonstrated the synthesis of carbon nanotube structures with different functional groups localized to the ends of the nanotube versus the sidewalls of the nanotube. However, the brief recipe provided does not provide sufficient teaching to enable the strong localization of groups to the CNT end (a significant amount of sidewall functionalization by the end groups will occur utilizing HNO3 etching chemistries of the sort described by this work). Furthermore, these recipes are not general and not directed to the synthesis of Rf-CNB materials for directed self-assembly applications nor are they suitable thereto, which places different constraints upon the chemistry for useful embodiment.

Carbon Nanotube Spaceframe Materials

Carbon nanotube based spaceframe materials & their composites are a novel class of materials which engender a wide range of superior materials properties.

Carbon nanotube spaceframe based materials are a general class of materials formed of networked lattice of carbon nanotubes connected together in an end-to-end configuration.

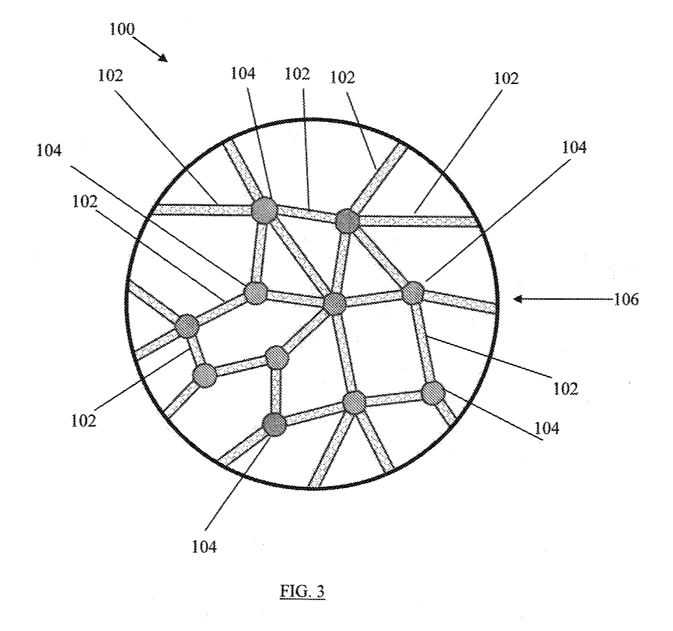

As illustrated in FIG. 3, a embodiment 100 of carbon nanotube spaceframe based materials is disclosed. They are constructed from carbon nanotubes 102 connected together end-to-end by nodes 104 comprised of non-graphitic materials to form a networked lattice 106. The assembly of Regiofunctional Carbon Nanotube Beams (Rf-CNBs) by crown-to-crown connection into nodes to form a networked lattice configuration is a useful embodiment of these materials. For Rf-CNB spaceframe materials with crowns of carbonizable polymeric materials, the spaceframe network can be carbonized to form a purely graphitic spaceframe network.

The range of useful properties which can be achieved by spaceframe-type materials can be enhanced by materials processing used to convert the molecular structure of the nodes from the structures formed by the self-assembly process, which is referred to as node conversion, into other molecular forms. The molecular form of the nodes typically created by self-assembly processes consists of a relatively soft, visco-elastic structure comprised of physically entangled polymeric chains (linear or branching). Use of suitably chosen crown materials and processing conditions the molecular structure of the nodes can be converted to a variety of secondary forms. These secondary forms can include cross-linked polymers, carbonized, graphitized, ceramic, diamond-like along with tailored functionalization.

With regard to the cross-linked polymer secondary form, the polymer materials which form the crowns can be cross-linked to create more rigid and stable mechanical materials. To create spaceframes with cross-linked polymeric nodes it is typically advantageous to utilize crown-materials which are amenable to cross-linking (e.g. polymers with unsaturated sites). Cross-linking of such polymers can be achieved by various means including radical initiation, vulcanization, and similar methods.

When creating carbonized and graphitized secondary forms, for appropriately chosen polymeric crowns (e.g. polybutadienes, nitriles, cellulosic polymers, etc.) pyrolysis of these materials can be used to convert the polymer chains into polycyclic aromatic carbon sheets or well-ordered graphitic carbons. Carbonization and full graphitization are two extremes of a spectrum of molecular structures created by pyrolysis. Appropriately chosen catalysts such as transition metals, boron, and like catalysts are useful for achieving graphitization under milder reaction conditions. For node conversion it is beneficial to be in the form of atomic additives and opposed to nanoparticles. The relatively small size of the nodes enables atomic catalyst to be effective at catalyzing node graphitization without coalescing to form nanoparticles. These catalysts can be introduced into the nodes either by direct inclusion into the crown polymer structure through species such as step-polymerizable metalorganics (e.g. 1,1′-dicarboxyferrocene), by the dissolving metalorganics into the nodes or by similar methods.

Additionally, nodes can be converted to hard ceramic materials by the inclusion of secondary atomic species into the molecular structure, e.g. boron, silicon, etc. These species can be included by mechanisms such as hydroboration at unsaturation sites; use of silicon bearing polymers in the Rf-CNB crowns, dissolution of silicon containing organics into the nodes, etc. Heat treatment can then be used convert these structures into carbon silicides, carbon borides, or similar hard-ceramic materials.

Further, the incorporation of polymers such as poly(hydridocarbyne) into the nodes can enable conversion of the node carbon into diamond-like structures through processing methods as detailed in the literature.

Still further, the polymeric materials forming the crowns can be functionalized to incorporate secondary functionalities tailored to create specific properties in the nodes. Examples of these functionalities include sites capable of creating reversible cross-linking such as carboxylic sites which can undergo reversible cross-linking in the presence of divalent ions; thiol sites which are capable of creating reversible di-sulfide cross-links; moieties which tailor the glass-transition temperature of the node and environmentally sensitive moieties, such as complexing agents such as porphyrins. These functionalities can readily be introduced at unsaturation sites in crown-polymer materials. These can be introduced either before or after self-assembly.

It is noted that polybutadiene based crown materials are well suited to enable all of the node-conversions described except for diamond-like materials.

In another embodiment, a carbon nanotube spaceframe which coexists with a fluid phase to form a bicontinuous matrix structure forms a non-static spaceframe lattice structure. In such a structure the nodes are formed from non-graphitic structures with a composition such that the forces linking the nanotubes at the nodes are quasi-reversible in solution such that the node connections can be formed and unformed without hindering the nanotube structure's ability to continue to associate to form nodes. For example, an Rf-CNB with water solubilized sidewalls and water insoluble crowns can be used in water solution to form spaceframe structures which are quasi-stable, but which will respond to disruption (e.g. by a sudden impulsive force) by “healing” itself into a new lattice after the disrupting force is removed. Or, in another embodiment, the sidewalls might be made water soluble and the crowns only moderately water insoluble; in this case the spaceframe lattice maintains a constant state of flux, with connections always forming and un-forming in solution to create a highly viscous “fluid” system in which the properties are strongly modified by the presences of a non-static spaceframe.

In order to form a networked lattice each node connects, on average, at least 3 nanotubes (or at least 4 nanotubes for 3D space-filling lattices). In many embodiments carbon nanotube spaceframes will have many more connections per node than this.

In order to accommodate this requirement, the volume of the node points must be large enough to provide sufficient surface area to connect to the desired number of carbon nanotubes. Therefore the minimum node volume can be estimated as being approximately Vnode≧(4/3)*π*(rnanotube)3. Increased node size can be used to confer increased binding energy of the nodes.

Extra material can be added to the node volume which is not directly bonded to the ends of the nanotube to alter the physico-chemical properties of the node. E.g. with hydrophobic crowns in aqueous solution, hydrophobic molecules such as aromatics or alkanes can be added to the system; these will preferentially segregate into the crowns changing the effective size of the crown and the crown-solution interaction.

In a carbon nanotube spaceframe materials herein described the volume occupied by the nodes is less than 50% of the total volume of the material architecture.

In some instances it is beneficial to form networks with relatively low connection number to form continuous networks of low topological dimension termed “hyperbranched networks.” For these networks the average connection number per node will be greater than 2 and typically around 3.

Small amounts of catalytic species (e.g. iron, nickel, cobalt, boron, etc.) can be added to catalyze the carbonization of spaceframe lattice nodes to an all graphitic lattice.

Functional groups can be attached to the carbon nanotube spaceframe structures to modify the surface properties of the spaceframe lattice (e.g. for utilization in bicontinuous composites).

Similar arrangements of matter with rods made of materials other than carbon nanotubes held together by a different material at the nodes is also considered in this invention.

An example of this would be carbon nanofibers, metallic nanorods/nanowires/whiskers, semiconductor nanowires joined by polymeric nodes to form a networked lattice.

Previous chemistries have been reported which chemically link nanotubes together. The method herein reported is distinguished from these chemistries by the form and structure of the material produced. Previous methods have produced “rings,” “end-to-end” nanotube linkage, “end-to-side” nanotube linkage, and “stars” of nanotubes linked to a central dendrimer structure. Fundamentally these methods are distinct in that they have never been demonstrated or even speculated to form lattice network materials.

Ring, and other previously reported end-to-end linked structures are distinct from spaceframe type structures in that they consist only in the linkage of two nanotube ends together. These linkages have been formed by difunctional groups which are short relative to the diameter of the nanotube which will necessarily result mostly in linkages between only two nanotube ends. This is insufficient to form a lattice network which are formed by regioselected end-to-end linkage of at least three nanotubes per node or on at least average greater than two nanotubes per node leading to hyperbranched extensive networks.

Dendrimer “star” formations (and similar materials created by tethering nanotubes to a central nanoparticle) are distinguished from the carbon nanotube spaceframe material herein described by the lack of spatial extent and/or network possessed by the dendrimer star structures. That is, under properly chosen conditions, dendrimer based spaceframe structures could possibly be created; however, in previous dendrimer work these structures are not achieved nor are they even considered as a possibility. In the cases where nanotubes bridge between dendrimer stars the bridging is a random occurrence and does not serve to create a superstructure.

In the prior art, material is described that consists of shortened, single-walled carbon nanotubes connected by end-functionalization and without sidewall functionalization. Those limitations of that material make it less useful than the material described herein. The variable length allows this material to possess more general usefulness to address a wider range of problems while the use of multiwalled carbon nanotubes enables this material to be synthesized more economically than one based on single-walled nanotubes. Furthermore, the utilization of a different type of end-to-end linkage enables different materials properties to be obtained (e.g. carbonized networks) while the potential incorporation of sidewall functionalization enables this material to be tailored to more facile use in composite materials.

Carbon Nanotube Spaceframe-Based Composite Materials

Composite materials incorporating CNT spaceframe elements (as described above) are also expected to demonstrate advanced useful materials characteristics.

One form of CNT-spaceframe composite materials are bicontinuous matrix composites, in which the spaceframe coexists with another matrix material to form a fully reticulated bicontinuous composite structure. This type of composite structure can be formed with polymeric, metallic, or ceramic materials forming the second matrix phase.

Another form of CNT-spaceframe composite material is formed by the conformal coating of material onto the surface area of the spaceframe structure. For instance, metal and ceramic materials can be conformally deposited onto graphenic nanotube surfaces by vapor methods such as atomic layer deposition. Coating of such materials can be advantageous to increase materials properties such as the stiffness, strength and conductivity of the material.

Another form of CNT-spaceframe composite material is the use of spaceframe material as matrix for other filler materials such as carbon fiber in place of more traditional epoxy matrixes. Alternatively a bicontinuous spaceframe-polymer matrix (or other bicontinuous spaceframe matrix materials) could be used to form the matrix for filler materials.

It should be noted that spaceframe materials can also be used in a particulated or discontinuous form as a filler material in a secondary matrix to improve materials properties. Such a particulated spaceframe object may take the form of a small particle of bicontinuous matrix material which can act as inclusions within an overall matrix to modify the materials properties of the composite. Alternately the spaceframe particle may be devoid of a secondary reticulated matrix.

Carbon Nanotube Spaceframe Synthesis

Assembly of carbon nanotube spaceframe materials from Rf-CNB precursors is achieved by the transport of Rf-CNBs through suspension in a fluid phase followed by selective precipitation of the crown end groups. This can be achieved either through true “dissolution” of both crown and end groups followed by a modification to the solution system leading to a selective insolubilization of the crowns, or by thixotropically driven (commonly ultrasonic or ultra-high shear fluidization) suspensions of Rf-CNBs into solvents in which the sidewalls are soluble and the crowns are not, followed by a cessation of the thixotropic driving force.

In an embodiment of this process the self-assembly process is mediated by reversible association forces in solution (e.g. hydrophilic/hydrophobic coalescence, zeta potential, acid/base associations, etc.). In general these forces will depend upon and be modified by the environmental conditions of the solvent (solvent composition, pH, etc.). These parameters can be modified to selectively insolubilize different regions in the Rf-CNBs to direct self-assembly of these materials. While the self-assembly in this embodiment is mediated by reversible forces, the forces linking the nodes and network together can be modified after assembly to include irreversible bonding forces by processes such as cross-linking the node materials.

Secondary forces can be applied to assist the self-assembly process. Forces may be applied to affect the process by interaction with the nanotubes, the crowns or the solvents or a combination of these. An example of this type of force could be the application of an electric field to induce a mutually aligning force to the nanotubes.

In many cases, especially in the case of hydrophobic crown self-assembly, the zeta potential (as mentioned above) is particularly important to the dynamics of self-assembly. The zeta potential of the crowns acts to produce a force-at-a-distance between the crowns (for two like crowns this force is always repulsive in sign). The zeta potential can be modified by species both present in solution (e.g. acids, bases, salts, surfactants, etc.) and by moieties comprising the surface of the crown itself. In aqueous solution, zeta potentials of strongly hydrophobic surfaces are typically negative owing to the preferential adsorption of hydroxide ions from solution. This potential can be modified via the incorporation of terminal moieties. E.g. carboxyl, hydroxyl, methoxy-ester, ether, amides, amines (primary, secondary, or tertiary), etc. In particular, the incorporation of positively charged groups onto the crown surface (such as amines) can be utilized to counteract the negative charge typically associated with the surface. The optimal surface density of amines utilized to create a zeta potential near zero will depend upon the surface charge density of the rest of the surface. This can be measured directly for macroscopic films of equivalent composition by common techniques used to measure the zeta potential of surfaces and films (streaming potential measurement).

While the invention has been described with a certain degree of particularity, it is manifest that many changes may be made in the details of construction and the arrangement of components without departing from the spirit and scope of this disclosure. It is understood that the invention is not limited to the embodiments set forth herein for purposes of exemplification, but is to be limited only by the scope of the attached claims, including the full range of equivalency to which each element thereof is entitled.

We claim:

1. A method to create an enhanced carbon nanotube spaceframe network which contains an assembly of regiofunctional carbon nanotube beams by crown-to-crown connection into nodes to form a networked lattice configuration, said method comprising the following steps:
selecting crown materials
applying processing conditions, resulting in the production of secondary forms

2. The method of claim 1 wherein said step of selecting crown materials is defined as selecting crown materials from the group consisting of polymers with unsaturated sites, polymeric crowns, silicon boron, poly(hydridocarbyne).

3. The method of claim 1 wherein said step of applying processing conditions is defined as applying processing conditions from the group consisting of radical initiation, vulcanization, pyrolysis, hydroboration at unsaturation sites, using silicon bearing polymers in the Rf-CNB crowns, dissolution of silicon containing organics into the nodes and poly(hydridocarbyne)

4. The method of claim 1 wherein said secondary forms are defined as secondary forms selected from the group consisting of cross-linked polymers, carbonized, graphitized, ceramic, diamond-like along with tailored functionalization.

5. The method of claim 1 wherein said crown materials is defined as polymers with unsaturated sites, the process is defined as radical initiation and the secondary form is defined as a cross-linked polymer secondary form.

6. The method of claim 1 wherein said crown materials is defined as polymers with unsaturated sites, the process is defined as vulcanization and the secondary form is defined as a cross-linked polymer secondary form.

7. The method of claim 1 wherein said crown materials is defined as polymeric crowns, the process is defined as pyrolysis and the secondary form is defined as a graphitized secondary form.

8. The method of claim 1 wherein said crown materials is defined as polymeric crowns, the process is defined as pyrolysis and the secondary form is defined as a carbonized secondary form.

9. The method of claim 1 wherein said crown materials is defined as including boron, the process is defined as hydroboration at unsaturation sites and the secondary form is defined as a ceramic secondary form.

10. The method of claim 1 wherein said crown materials is defined as including silicon, the process is defined as using silicon bearing polymers in the Rf-CNB crowns and the secondary form is defined as a ceramic secondary form.

11. The method of claim 1 wherein said crown materials is defined as including silicon, the process is defined as dissolution of silicon containing organics into the nodes and the secondary form is defined as a ceramic secondary form.

12. The method of claim 1 wherein said crown materials is defined as including poly(hydridocarbyne) and the secondary form is defined as a diamond-like structure secondary form.
